(19) United States
(12) Patent Application Publication
(10) Pub. No.: US 2001/0036937 A1
(43) Pub. Date: Nov. 1, 2001

(54) Treatment of ICU-associated hypocalcemia with vitamin D compounds

(76) Inventors: Leticia Delgado-Herrera, Riverwoods, IL (US); Patricia Loreen Mershimer, Wilmette, IL (US); Richard Zager, Seattle, WA (US)

Correspondence Address: Steven F. Weinstock, ABBOTT LABORATORIES, D-377/AP6D-2, 100 Abbott Park Road, Abbott Park, IL, 60064-6050, US

(21) Appl. No.: 09/827,495

(22) Filed: Apr. 6, 2001

Related U.S. Application Data
(60) Provisional application No. 60/195,853, filed on Apr. 7, 2000.

(51) Int. Cl. A61K 31/59

(52) U.S. Cl. 514/167

(57) ABSTRACT

The present invention relates to a method of improving intensive care unit (ICU)-associated hypocalcemia in a mammal by the administration of a vitamin D compound, or other compounds exhibiting vitamin D-like activity, to the mammal for a sufficient period of time to improve or restore the serum calcium levels of the mammal.

This application claims priority to provisional application Ser. No. 60/195853, filed Apr. 7, 2000.

### TECHNICAL FIELD

The present invention relates to the treatment of hypocalcemia in mammals, and more particularly to a method of improving intensive care unit (ICU)-associated hypocalcemia in a human by the administration of a vitamin D compound, or other compounds exhibiting vitamin D-like activity, to the mammal for a sufficient period of time to improve or restore the serum calcium levels to normal.

### BACKGROUND OF THE INVENTION

Although various biological functions for vitamin D compounds have been discovered, the role of vitamin D and other compounds having vitamin D-like activity on ICU related hypocalcemia has not been characterized.

A considerable body of published information indicates that hypocalcemia, as reflected by a significant reduction in the ionized serum calcium concentration, is a frequent complication of severe sepsis syndrome and multi-organ failure (MOF). Its potential implications for patient outcomes are suggested by the facts that: 1) its frequency and severity predict adverse patient outcomes (comparable to APACHE II scores); and 2) that patients are often times treated with large amounts of intravenous calcium salts to offset potential adverse effects (e.g., cardiac dysfunction, seizures etc).

Currently, hypocalcemia in an intensive care unit (ICU) setting is either not treated or it is treated only when the medical professional judges it to be of life threatening severity. Existing treatment modalities for hypocalcemia in this setting are limited to intravenous (IV) infusions of inorganic (e.g., CaCl₂) or organic (e.g., calcium gluconate) salts. The problems associated with the administration of calcium salts are: (a) IV calcium infusions have attendant risk of cardiotoxicity, (b) IV calcium infusions only treat the manifestations of the abnormality, i.e., low ionized calcium, not the metabolic cause of the abnormality, (c) because ICU-related hypocalcemia reflects a blood/tissue maldistribution of calcium and not a net calcium loss the administration of calcium may cause total calcium overload, and (d) calcium infusions are given as a “sliding scale” (increasing degrees of hypocalcemia relative to increasing doses of IV calcium).

The present inventors have determined that hypocalcemia occurs in over 75% of patients who are hospitalized in an intensive care unit (ICU) setting. This is true regardless of whether the patients are housed in a Medical, Surgical, Trauma, Neurosurgical or Burn ICU setting. The degree of hypocalcemia is variable, upwards to a 25-30% reduction is ionized serum calcium levels, more generally ranging from a 10% to 25% reduction in ionized serum calcium levels.

The present invention comprises a method for treating ICU-associated hypocalcemia in a mammal which comprises administering to the mammal an amount of vitamin D compound sufficient to improve the ionized serum calcium level of the mammal.

### SUMMARY OF THE INVENTION

It has now been found that in patients showing ICU-related hypocalcemia the administration of a vitamin D compound or a compound having vitamin D-like activity improves or maintains the ionized serum-calcium balance.

One aspect of the invention provides a method for treating ICU-related hypocalcemia in a patient to increase the ionized serum calcium level in said patient which comprises administering to said mammal an amount of a vitamin D compound sufficient to improve the ionized serum calcium level of said mammal.

Yet another aspect of the invention provides the method described above wherein the vitamin D compound is vitamin D₃ or vitamin D₂.

Preferably, the vitamin D compounds useful in the method of the invention are selected from 1,25-dihydroxy vitamin D₃ and 1,25-dihydroxy-19-nor ergocalciferol.

Yet a further aspect of the invention is the method described above wherein the vitamin D compound is administered in an amount of from about 0.1 micrograms to about 2 milligrams per day depending on the vitamin D compound administered.

An additional aspect of the invention is a method for minimizing the development of hypocalcemia in patients admitted to a hospital setting by (a) testing the patient to determine the ionized serum calcium level, and (b) administering to the patient an amount of a vitamin D compound sufficient to improve or maintain a normal ionized serum calcium level of the patient.

### DETAILED DESCRIPTION OF THE INVENTION

These terms shall have the following definitions when used throughout the specification and claims:

“Hypocalcemia” is defined as a reduction in the ionized calcium below the normal validated range for a given hospital laboratory. The methods for validation are well known in the clinical arts. Typically, the normal range (total calcium) is between about 9 and about 10.5 mg/dl for adults and about 8.8 and about 10.8 mg/dl for children. The normal range of ionized calcium is between about 4.5 and about 6.6 mg/dl for adults.

“ICU” or “Intensive Care Unit” means a designated unit or location where critically ill patients are treated or monitored. Typically, the critically ill patients are categorized as having ASA physical status 2, 3, or 4.

“ICU-related hypocalcemia” or “ICU-associated hypocalcemia” means hypocalcemia that occurs in patients hospitalized in an Intensive Care Unit setting.

As used herein the term “vitamin D compound” encompasses compounds which control one or more of the various vitamin D-responsive processes in mammals, i.e. intestinal calcium absorption, bone mobilization, bone mineralization, and cell differentiation. Thus the vitamin D compounds encompassed by this invention include cholecalciferol and ergocalciferol and their metabolites, as well as the synthetic cholecalciferol and ergocalciferol analogs which express calcemic or cell differentiation activity. Without limiting the vitamin D compounds encompassed by the present invention, these synthetic cholecalciferol and ergocalciferol analogs comprise such categories of compounds as the 5,6-trans-cholecalciferols and 5,6-trans-ergocalciferols the fluorinated cholecalciferols, the side chain homologated cholecalciferols and side chain homologated 22 cholecalciferols, the side chain-truncated cholecalciferols, the 19-nor cholecalciferols and ergocalciferols, and the 10,19-dihydovitamin D compounds.

Some specific examples of such compounds include vitamin D metabolites or analogs such as vitamin D₃, vitamin D₂, 1-hydroxyvitamin D₃, 1-hydroxyvitamin D₂, 1,25-dihydroxyvitamin D₃, 1,25-dihydroxyvitamin D₂, 25-hydroxyvitamin D₃, 25-hydroxyvitamin D₂, 24,24-difluoro-25-hydroxyvitamin D₃, 24,24-difluoro-,-dihydroxyvitamin D₃, 24-fluoro-25-hydroxyvitamin D₃, 24-fluoro-,-dihydroxyvitamin D₃, 2-fluoro-25-hydroxyvitamin D₃, 2-fluoro- -hydroxyvitamin D₃, 2-fluoro- ,25-dihydroxyvitamin D₃, 26,26,26,27,27,27-hexafluoro-25-hydroxyvitamin D₃, 26,26,26,27,27,27-hexafluoro-1,25-hydroxyvitamin D₃, 24-25-dihydroxyvitamin D₃, 14,25-trihydroxyvitamin D₃, 5,26-dihydroxyvitamin D₃, 15,26-trihydroxyvitamin D₃, 3,25-dihydroxyvitamin D₃, 23,25,26-trihydroxyvitamin D₃, and the corresponding 1-hydroxylated forms, 25-hydroxyvitamin D₃, -26,23-lactone and its 1-hydroxylated derivative, the side chain, nor, dinor, trinor and tetranor-analogs of 25-hydroxyvitamin D₃, and of ,-dihydroxyvitamin D₃, 1-hydroxypregnacalciferol, and its homo and dihomo derivatives, 1, -dihydroxy-24-20i-vitamin D₂, 24-homo-1,25-dihydroxyvitamin D₃, 24-dihomo-1,25-dihydroxyvitamin D₃, 24-trihomo-1,25-dihydroxyvitamin D₃, and the corresponding 26- or 26,27-homo, dihomo or trihomo analogs of 1,25,dihydroxyvitamin D₃, as well as the corresponding 19-nor compounds of those listed above.

The vitamin D compound can be administered by any means suitable to improve the ionized serum calcium level of the mammal. Preferably, the compound is administered via an intravenous (IV) injection.

The vitamin D compound can be formulated following techniques known in the art and suitable for administration via the selected route. For instance, oral capsules are disclosed in U.S. Pat. No. 4,341,774 and formulations suitable for IV administration are disclosed in U.S. Pat. No. 4,308,264 and WO 96/36340.

Preferably, the vitamin D compound is administered in a therapeutically effective amount of from about 0.1 microgram to about 2 milligrams per day depending upon the vitamin D compound administered. Also, the vitamin D compound is preferably administered daily to the mammal for about 1-4 weeks.

Materials and Methods

### EXAMPLE 1

Identification of Incidence of ICU-associated Hypocalcemia

Three patient groups were defined. Group A: ICU patients who were in a unit for >48 hours, or who died within the first 48 hours following admission to that unit; Group B: Non critically ill ICU controls: patients who were hospitalized in an ICU for <48 hrs, followed by their transfer either to home or to a general medical/surgical ward; and Group C: patients admitted to the general medical or surgical ward and who never required ICU admission.

Laboratory data and patient outcome for each group was collected. Laboratory values for Group A were recorded for the duration of time the patient remained in the ICU, up to a maximum of 5 days; Group B: for the duration of time the patient remained in the ICU (by definition, <48 hours); and Group C: for the first 48 hours of hospitalization. Data included ionized serum Ca, Mg, phosphate,, creatinine, arterial pH, and blood cultures. Serum albumin, liver enzymes, creatinine kinase and total Ca were available in less than ten percent of patients during the time period examined. Therefore, these data were not included in the analyses. Hypocalcemia was defined as an ionized calcium level of less than 1.16 mmol/L, with a normal range for this institution being 1.16-1.27 mmol/L.

Patient outcome was defined by mortality while in an ICU (Groups A,B) or on a hospital ward. One patient died after being admitted to the general ward from the ICU and this patient is not included in the mortality data.

The incidence of hypocalcemia was 88% in Group A patients, 66% in Group B patients, and 26% in Group C patients (p<0.001 amongst all groups; p<0.001 for each pairwise comparison). The incidence of hypocalcemia in Group A was irrespective of the admission diagnoses or the ICU to which the patients were admitted. The mean of the average ionized calcium levels in Group A was below the normal range (1.09 mmol/L), with the median of averages being 1.1 mmol/L (range 0.66-1.29 mmol/L). Amongst Group B patients, the mean of the average ionized calcium levels was barely below the normal Ca range (1.15 mmol/L) and the median of average level was 1.16 mmol/L (range 0.85-1.28 mmol/L). Group C patients had normal mean and median average calcium levels (each 1.21 mmol/L; ranges, 1.04-1.35 mmol/L).

### EXAMPLE 2

Model of Sepsis/MOF Associated Hypocalcemia

Using male CD-1 mice, a sepsis syndrome-like state is induced. There are a number of methods for inducing a sepsis syndrome-like condition. Three such methods are described below:

a) Injection of increasing doses of purified E. coli endotoxin (B6-026; administered IP). The doses which induce an LD 25-50 are defined. Once accomplished, the development of hypocalcemia is assessed at different time points, ranging from 8-48 hrs. Ionized calcium will be measured on blood withdrawn at time of sacrifice (obtained from the inferior vena cava under pentobarbital anesthesia). Ionized Ca values are measured using techniques well known in the art.

b) Injection of heat killed (boiled) E. coli (approximately 1×107 organisms). Previous studies (unpublished) indicate that administration of heat killed bacteria may allow for a more clinically relevant form of endotoxin loading without the risks of uncontrolled infection. The assessments of toxicity/hypocalcemia will be assessed as described above.

c) Model of non septic cytokine “storm” (i.e., non-infective ‘sepsis syndrome’). The induction of rhabdomyolysis recapitulates many of the aspects of multiorgan failure, including acute renal failure. This syndrome can be induced by intramuscular glycerol injection, and leads to increased TNF, and ‘downstream’ cytokine release. Hence, this model is used to recapitulate MOF-associated hypocalcemia. Because this model induces renal failure by 24 hrs (which can independently cause hypocalcemia), assessments of serum calcium (and phosphate concentrations) are made within the first 8 hrs post glycerol injection.

### EXAMPLE 3

Offsetting Hypocalcemia with Paricalcitol

Paricalcitol is also referred to as 19-nor 1,25 dihydroxy vitamin D₂ or 19-nor 1,25-(OH)₂D₂. The model of sepsis syndrome is used to test the efficacy of paricalcitol and/or other vitamin D derivative to correct hypocalcemia. 100-200 CD-1 mice are treated in two ways. One set is dosed with the test compound in order to prevent hypocalcemia (i.e. drug administration immediately prior to the induction of hypocalcemia). A second set is treated following induction of hypocalcemia. For instance intervention, administration of test compound, can occur 2-3 hrs post induction of hypocalcemia. The animals are monitored and aliquots of blood withdrawn for a period of time following administration, e.g., 0-48 hrs. The samples are measured to: 1) confirm that a given dose of vitamin D corrects the hypocalcemia without inducing hyperphosphatemia); 2) demonstrate that the therapy has no adverse effects, for example on renal function, histology, and/or evidence for tissue metastatic calcification; 3) determine whether prophylactic or therapeutic vitamin D administration can improve survival rates when an LD 50 dose of the precipitating challenge has been administered.

The study indicates that the test drug can be safely administered and can correct the hypocalcemic state.

### EXAMPLE 4

Treatment of ICU-associated Hypocalcemia

A plurality of hypocalcemic patients are identified and randomized to receive either a vitamin D compound or the vitamin D carrier (placebo group). The patients are administered the blinded agent on a daily basis and continue to receive routine therapy, including IV calcium salts as needed. The patients are monitored to determine whether 1) the need for IV calcium salts are decreased in order to maintain a normal serum ionized calcium; 2) vitamin D therapy completely eliminates the need for IV calcium supplementation; and 3) vitamin D therapy provides an improvements in patient outcomes, for example, incidence of morbid events, or decreased mortality rates.

Results

Based on the above studies and findings, it has been determined that a vitamin D compound may be utilized to positively modulate ICU-associated hypocalcemia.

What is claimed is:

1. A method for treating ICU-associated hypocalcemia in a mammal to restore or maintain ionized serum calcium in said mammal which comprises administering to said mammal a therapeutically effective amount of a vitamin D compound.

2. The method of claim 1 wherein the vitamin D compound is selected from compound having Formula I.

3. The method of claim 1 wherein the vitamin D compound is administered in an amount of from about 0.1 micrograms to about 2 milligrams per day depending upon the vitamin D compound administered.

4. The method of claim 1 wherein the vitamin D compound is administered daily to said mammal for about 1-4 weeks.

5. The method of claim 1 wherein the vitamin D compound is combined with a non-toxic pharmaceutically acceptable carrier prior to administration.

6. The method of claim 1 wherein the vitamin D compound is vitamin D₃ or a vitamin D₃ derivative.

7. The method of claim 6 wherein the vitamin D compound is 1,25-dihydroxy vitamin D₃.

8. The method of claim 1 wherein the vitamin D compound is vitamin D₂ or a vitamin D₂ derivative.

9. The method of claim 8 wherein the vitamin D₂ compound is 1,25-dihydroxy-19-nor ergocalciferol.
